(19) United States
(12) Plant Patent Application Publication
(10) Pub. No.: US 2016/0157403 P1
(43) Pub. Date: Jun. 2, 2016

(54) Stevia plant named `SW 107'

(50) ;

(71) Applicant: S&W Seed Company, Five Points, CA (US)

(72) Inventors: Clinton C. Shock, Ontario, OR (US); Cheryl A. Parris, Ontario, OR (US)

(73) Assignee: S&W Seed Company, Five Points, CA (US)

(21) Appl. No.: 14/544,136

(22) Filed: Nov. 30, 2014

(51) Int. Cl. A01H 5/00

(52) U.S. Cl. PLT/226; PLT/258

(57) ABSTRACT

A new and distinct Stevia rebaudiana plant named ‘SW 107’ is described. The new variety is distinguished from other Stevia varieties by high productivity, including superior plant vigor and excellent overwintering capability; very sweet leaf taste with low bitterness and aftertaste and high rebaudioside A content.

### GENUS AND SPECIES

Stevia rebaudiana.

### VARIETY DENOMINATION

SW 107

### BACKGROUND OF THE INVENTION

Stevia rebaudiana is a plant species in the sunflower (Asteraceae) family, which has naturally sweet leaves. The active compounds that impart the sweet flavor to stevia leaves are steviol glycosides. The most common steviol glycosides are stevioside and rebaudioside A, both of which have up to 250 to 400 times the sweetness of sugar. Stevioside tends to be more prevalent in unimproved lines of stevia leaves than rebaudioside A. The development of new varieties of Stevia rebaudiana with higher levels of rebaudioside A than stevioside is desirable for use as a sugar substitute.

The present invention relates to a new and distinct variety of Stevia rebaudiana, the ‘SW 107’ line, which has a much higher concentration of rebaudioside A than stevioside as well as high plant vigor, excellent overwintering, high plant productivity, late flowering (an advantage for leaf production), and very sweet leaves with low bitterness and aftertaste.

The ‘SW 107’ line was derived in 2012 from a single plant in a population of recurrently selected Stevia rebaudiana, the population of which was originally grown from seed in a field in Chowchilla, Calif., USA. The plant that produced the ‘SW 107’ line was selected from its population based upon the following characteristics, which were measured and/or observed between 2012 and 2014.

Plant Vigor: the ‘SW 107’ line had greater growth, superior overwintering, and later flowering in comparison to the majority of the plant population from which the line was derived.

Leaf Taste: The leaves of the ‘SW 107’ line were found to have a strong sweet taste in comparison to a majority of the plant population from which the line was derived and distinctly less bitterness and aftertaste.

Steviol glycoside content: Initial analysis of rebaudioside A in the ‘SW 107’ line was found to be approximately 60% above the generalized rebaudioside A content of the plant population from which the line was derived.

Upon initial selection, the ‘SW 107’ line was asexually reproduced from rooted cuttings in May 2012. Plant yield and leaf quality of the ‘SW 107’ line were confirmed in April through October 2014 through replicated yield trials in the following four locations in California and Oregon: Yuma CA; Hanford Calif.; Indio Calif.; and Ontario Oreg. At each of the four locations, the performance of the ‘SW 107’ line grown from cuttings was compared to seedlings grown from bulk commercial seed (“the check variety”). The following table compares various characteristics of the ‘SW 107’ line with the check variety.

TABLE 1

| | SW 107 | Check Variety |
|---|---|---|
| Percentage of Rebaudioside A | 10.39% | 6.85% |
| Percentage of Stevioside | 4.76% | 6.42% |
| Percentage of total Steviol | 16.89% | 14.71% |
| Glycosides | | |
| Ratio of Rebaudioside | 2.33 | 1.11 |
| A:Stevioside | | |
| Percentage of Rebaudioside A to | 61.8% | 46.8% |
| total Steviol Glycosides | | |

The data in the foregoing table shows that in comparison to the check variety, the ‘SW 107’ line contained more rebaudioside A (10.39% vs. 6.85%); less stevioside (4.76% vs. 6.42%); more total steviol glycosides (16.89% vs. 14.71%); a higher rebaudioside A to stevioside ratio (2.33 vs. 1.11); and a higher percentage of rebaudioside A to total steviol glycosides (61.8 vs. 46.8%). In addition to the foregoing, the ‘SW 107’ line also produced 40% more leaf yield than the check variety (4,511 lb of leaf per acre per year vs 3,216 lb of leaf per acre per year) based on yield results averaged over four locations and two harvest strategies (one or two cuttings per year). The higher yield of the ‘SW 107’ line was due in part to prolific branching from the plant base and low stand loss from Fusarium and other root diseases that reduced plant stand of the check variety.

### BRIEF SUMMARY OF THE INVENTION

The following characteristics of the new ‘SW 107’ line have been repeatedly observed and can be used to distinguish the ‘SW 107’ line as a new and distinct cultivar of a Stevia plant:

1. High plant vigor;

2. Excellent overwintering;

3. High plant productivity;

4. Late flowering;

5. Intense sweet flavor to the leaves with low bitterness and aftertaste;

6. Rebaudioside A content of 10.4%;

7. Stevioside content of 4.8%;

8. Total Steviol Glycoside content of 16.9%;

9. 62% Rebaudioside to total Glycoside Steviol; and

10. Over 4500 lb. of leaf per acre per year.

### BRIEF DESCRIPTION OF THE DRAWINGS

The accompanying photographs show, as nearly true as is possible in color illustrations of this type, typical flower and foliage characteristics of the ‘SW 107’ line. The plants depicted in the accompanying photographs are approximately one year old.

FIG. 1 depicts a color photograph of a fully mature ‘SW 107’ Stevia plant from roots to top of the plant near the end of the plant's first growing season.

FIG. 2 depicts a color photograph of ‘SW 107’ leaves.

FIG. 3 depicts a color photograph of an ‘SW 107’ leaf, including the shape and dimensions.

FIG. 4 depicts a color photograph of the branches, without leaves, of an ‘SW 107’ Stevia plant.

FIG. 5 depicts a color photograph of the flowers of an ‘SW 107’ Stevia plant.

### DETAILED BOTANICAL DESCRIPTION

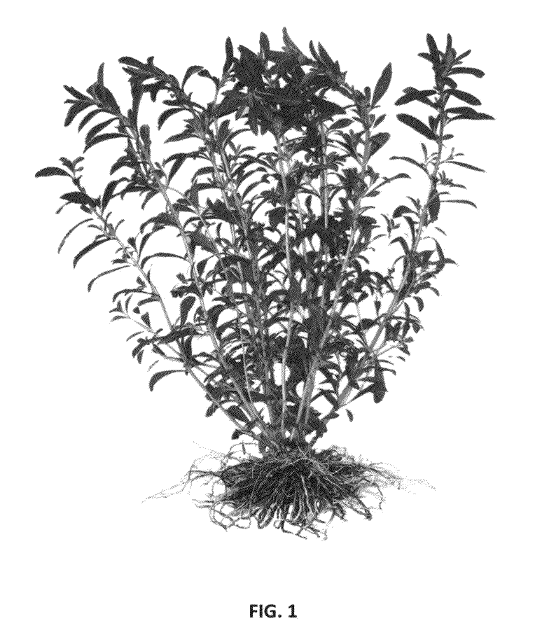

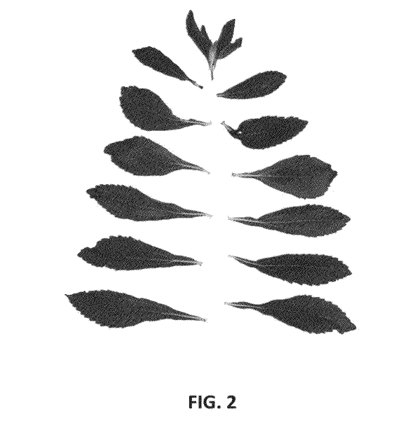

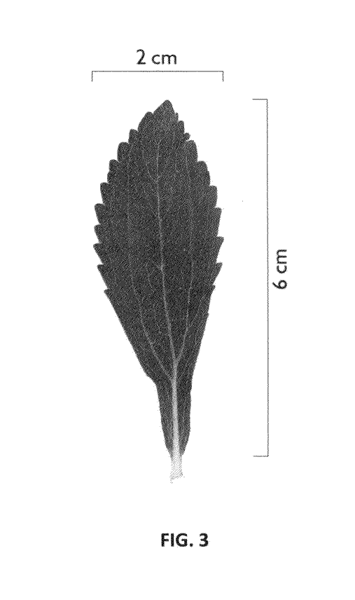

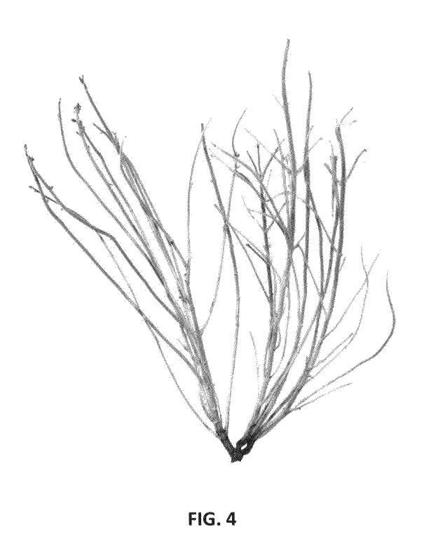

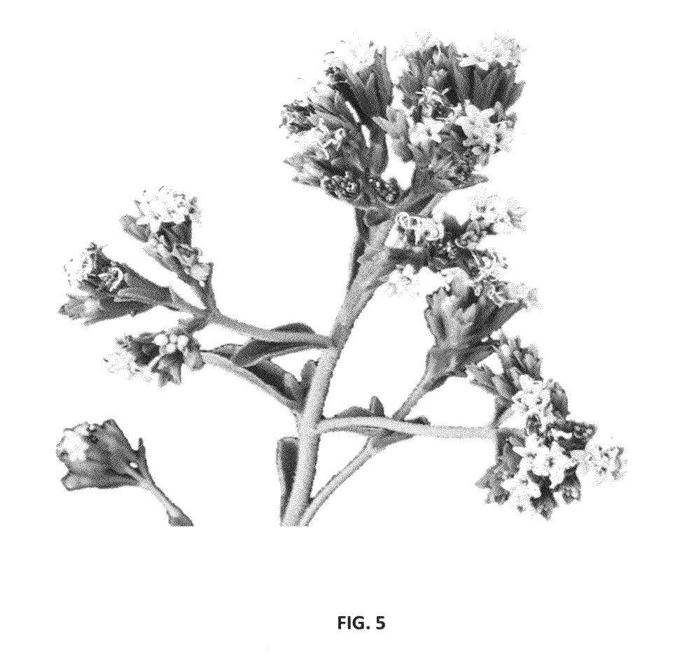

Following is a detailed botanical description of a new and distinct variety of a Stevia plant known as ‘SW 107.’ Plant observations were made on Aug. 21, 2014, on mature one year old plants grown in a field in Ontario, Oreg., USA in natural light. The ‘SW 107’ line has not been observed under all possible environmental conditions. Where characteristics, such as dimensions, sizes, colors, and other such characteristics are given, it is to be understood that such characteristics are approximations or averages and that such approximations are not intended to be limiting. It is also to be understood that the phenotype of the variety may be altered by changes with environment, such as season, temperature, light quality, light intensity, day length, cultural conditions, and the like. Color notations are based on The Royal Horticultural Society Colour Chart, The Royal Horticultural Society, London, 2007.
Botanical classification: Stevia rebaudiana cultivar SW 107.
Parentage: Seeds of Clinton C. Shock and stevia plants grown from the seeds in Chowchilla, Calif.
Plant (description based on FIG. 1):
   Type.—Perennial in climates with mild winter temperatures.
   Form and habit.—Multiple branching bush.
   Height (from top of soil).—44.5 cm to 1 m.
   Width (including flowers).—40.5 cm to 1 m.
   Number of nodes on main stem.—Variable; after initial growth, the number of principle stems proliferate.
   Number of basal buds.—Many.
   Harvest cycle.—Summer and fall or fall.
Leaves (description based on FIGS. 2 and 3):
   Length.—4.0 cm-7.5 cm.
   Width.—1.5 cm-4.5 cm.
   Arrangement.—Opposite.
   Shape.—Lanceolate.
   Apex descriptor.—Acute.
   Base descriptor.—Cuneate.
   Margin descriptor.—Serrate.
   Color, mature foliage.—Upper surface. RHS137C. Lower surface. RHS137C.
   Color, veins.—RHS 139C.
   Texture, mature foliage.—Upper surface. Slight pubescence. Lower surface. Moderate pubescence.
   Petiole length.—˜2 cm.
   Petiole diameter.—˜0.02 cm.
   Petiole texture.—Upper surface: Slight pubescence. Lower surface: Slight pubescence.
   Pubescence color.—Upper surface. RHS 139C. Lower surface. RHS 139C.
   Stevioside content.—4.76%.
   Rebaudioside A content.—10.39%.
   Rebaudioside A as a percentage of total steviol glycosides.—61.80%.
   Ratio of rebaudioside A to stevioside.—2.33.
   Total steviol glycosides.—16.89%.
   Leaf harvest period.—Summer plus fall or fall at the locations tested.
Stem (description based on FIG. 4):
   Length (excluding inflorescence).—Varying to 1 m.
   Diameter.—Varying to 1 m.
   Internode length.—Varies.
   Texture.—Pubescent.
   Color.—RHS 145B.
Flowers (description based on FIG. 5):
   Inflorescence type.—Panicle.
   Petal color.—RHS N155D.
   Petal length.—0.6 cm.
   Petal width.—0.1 cm.
   Bloom period.—October to November at Ontario, Oreg.
   Arrangement.—Composite.
   Shape.—Cluster.
   Fragrance.—None.
   Number of flowers per capitulum.—5.
Peduncle:
   Color.—RHS 145B.
   Length (cm).—Varies.
   Diameter.—˜0.15 cm.
   Aspect.—Perpendicular.
   Texture.—Pubescent.
Pedicles:
   Color.—RHS 145B.
   Length (cm).—Varies.
   Diameter.—˜0.03 cm.
   Aspect.—45 degrees.
   Texture.—Pubescent.
Disc florets:
   Arrangement.—Radially Symmetrical.
   Shape.—Tubular.
Phyllaries:
   Quantity.—5.
   Length.—˜0.7 cm.
   Width.—0.15 cm.
   Shape.—Lanceolate.
   Apex.—Acute.
   Base.—Fused.
   Margin.—Overlapping.
   Color.—RHS 145B.
Reproductive organs:
   Androecium.—Stamen quantity. 5. Anther length. ˜0.4 cm. Anther color. RHS 199A.
   Gynoecium.—Pistil length. ˜0.8cm. Style color. RHS N155B. Stigma color. RHS N155B.
Fruit and seed set:
   Flowering.—Later than most stevia lines.
   Seeds.—Small black achene with pappus attached typical of Stevia rebaudiana.
Cold hardiness: Hardy to 23° F. (−5° C.) at Ontario, Oreg.
Heat tolerance: Hardy to a monthly average maximum temperature of 107° F. (42° C.) at Yuma, Ariz., and Indio, Calif.

We claim:

1. A new and distinct cultivar of Stevia plant named ‘SW 107’ as substantially shown and described herein.
